(19) United States
(12) Patent Application Publication
(10) Pub. No.: US 2014/0352090 A1
(43) Pub. Date: Dec. 4, 2014

(54) ABSORBENT ARTICLES COMPRISING A FRAGRANCE ACCORD

(71) Applicant: The Procter & Gamble Company, Cincinnati, OH (US)

(72) Inventors: Nancy L. SCHUCHTER, Cold Spring, KY (US); Ann Marie FREY, Cedar Grove, IN (US); Megan E. JOHNS, Hyde Park, OH (US); Marianne MALMBAK-SCHAEFER, Kronberg (DE); Jason John OLCHOVY, West Chester, OH (US)

(73) Assignee: The Procter & Gamble Company, Cincinnati, OH (US)

(21) Appl. No.: 14/291,114

(22) Filed: May 30, 2014

Related U.S. Application Data
(60) Provisional application No. 61/829,505, filed on May 31, 2013.

(51) Int. Cl. A61K 8/02; A61Q 19/10; A61K 8/33; A61K 8/35; A61K 8/34; A61Q 13/00; A61K 8/37

(52) U.S. Cl. CPC A61K 8/0208; A61Q 13/00; A61Q 19/10; A61K 8/37; A61K 8/35; A61K 8/34; A61K 8/33; A61K 8/347; A61K 8/342
USPC 15/104.93

(57) ABSTRACT

A first absorbent article has a first fragrance accord and a second absorbent article has a second fragrance accord. The first fragrance accord includes 10% to 15% of a perfume having a vapor pressure of greater than 0.08 Torr at 25° C.; 35% to 65% of a perfume having a vapor pressure from 0.006 Torr at 25° C. to 0.08 Torr at 25° C.; 20% to 30% of a perfume having a vapor pressure of less than 0.006 Torr at 25° C. The second fragrance accord includes 10% to 20% of a perfume having a vapor pressure of greater than 0.08 Torr at 25° C.; 20% to 30% of a perfume having a vapor pressure from 0.006 Torr at 25° C. to 0.08 Torr at 25° C.; 45% to 70% of a perfume having a vapor pressure of less than 0.006 Torr at 25° C.

### FIELD

The present disclosure relates to absorbent articles, and more particularly, to absorbent articles comprising various fragrance accords.

### BACKGROUND

Absorbent articles, such as diapers and wet wipes, may include fragrances. For some consumers, the perceived efficacy of an absorbent article in terms of cleaning and ability to maintain and/or improve skin health may be affected by the scent or lack thereof of the absorbent article. Additionally, the scent of the absorbent article may affect the overall diaper changing experience for a consumer. However, different consumers perceive fragrance differently. For example, some consumers perceive a scented wipe that is able to mask the scent of a soil such as urine or feces as being able to clean well. The same type of consumer may also perceive a scented wipe that leaves a scent on the skin of a baby and/or the consumer as being able to clean well. Other consumers may negatively perceive a wipe that leaves a long-lasting scent on the skin of the baby and/or the consumer. The same type of consumer may perceive a wipe having a relatively low level of scent during use as being able to clean better than a wipe having no scent, while being gentler than a wipe having a higher level of scent.

Consumers may have similar perceptions for diapers as they do for wipes. For example, consumers may perceive a diaper that is able to mask the scent of urine or feces as being able to capture urine and feces well, while also maintaining and/or improving skin health. The same type of consumer may perceive a diaper that leaves a scent on the skin of a baby after use as being able to capture urine and feces well, while also maintain and/or improving skin health. Other types of consumers may negatively perceive a diaper that leaves a long-lasting scent on a baby. The same type of consumer may perceive a diaper that has a relatively low level of scent during use as being able to capture urine and feces than a diaper having no scent, while being gentler than a diaper having a higher level of scent.

Therefore, it would be beneficial to provide absorbent articles with a variety of fragrance compositions that meet the needs of various different consumers.

### SUMMARY

The present disclosure includes an array of absorbent articles comprising a first absorbent article having a first fragrance accord. The first fragrance accord may comprise from about 10% to about 15%, by total weight, of a perfume raw material having a vapor pressure of greater than 0.08 Torr at 25° C.; from about 35% to about 65%, by total weight, of a perfume raw material having a vapor pressure from 0.006 Torr at 25° C. to 0.08 Torr at 25° C.; and from about 20% to about 30%, by total weight, of a perfume raw material having a vapor pressure of less than 0.006 Torr at 25° C. The array comprises a second absorbent article having a second fragrance accord. The second fragrance accord may comprise from about 10% to about 20%, by total weight, of a perfume raw material having a vapor pressure of greater than 0.08 Torr at 25° C.; from about 20% to about 30%, by total weight, of a perfume raw material having a vapor pressure from 0.006 Torr at 25° C. to 0.08 Torr at 25° C.; and from about 45% to about 70%, by total weight, of a perfume raw material having a vapor pressure of less than 0.006 Torr at 25° C.

In some exemplary configurations, an array of absorbent articles may include a third absorbent article having a third fragrance accord. The third fragrance accord may comprise from about 45% to about 70%, by total weight, of a perfume raw material having a vapor pressure of greater than 0.08 Torr at 25° C.; from about 10% to about 25%, by total weight, of a perfume raw material having a vapor pressure from 0.006 Torr at 25° C. to 0.08 Torr at 25° C.; and from about 20% to about 30%, by total weight, of a perfume raw material having a vapor pressure of less than 0.006 Torr at 25° C.

Aspects of the present disclosure include an array of absorbent articles comprising a first absorbent article having a first fragrance accord. The first fragrance accord may comprise from about 10% to about 15%, by total weight, of a perfume raw material having a Kovats Index of less than 1300; from about 35% to about 65%, by total weight, of a perfume raw material having a Kovats Index from 1300 to 1450; and from about 20% to about 30%, by total weight, of a perfume raw material having a Kovats Index of greater than 1450. The array comprises a second absorbent article having a second fragrance accord. The second fragrance accord may comprise from about 10% to about 20%, by total weight, of a perfume raw material having a Kovats Index of less than 1300; from about 20% to about 30%, by total weight, of a perfume raw material having a Kovats Index from 1300 to 1450; and from about 45% to about 70%, by total weight, of a perfume raw material having a Kovats Index of greater than 1450.

In some exemplary configurations, an array of absorbent articles may include a third absorbent article having a third fragrance accord. The third fragrance accord may comprise from about 45% to about 70%, by total weight, of a perfume raw material having a Kovats Index of less than 1300; from about 10% to about 25%, by total weight, of a perfume raw material having a Kovats Index from 1300 to 1450; and from about 20% to about 30%, by total weight, of a perfume raw material having a Kovats Index of greater than 1450.

The first absorbent article may be a first diaper and the second absorbent article may be a second diaper. The first absorbent article may be a first wipe and the second absorbent article may be a second wipe. The first wipe may comprise a first lotion composition and the second wipe may comprise a second lotion composition, wherein the first lotion composition comprises the first fragrance accord, wherein the second lotion composition comprises the second fragrance accord. The first and second wipes may each comprise a non-woven substrate. The first and second wipes may each comprise a co-form structure.

Aspects of the present disclosure include a wet wipe comprising a substrate and a lotion composition, wherein the lotion composition comprises a fragrance accord. The fragrance accord comprises from about 10% to about 15%, by total weight, of a perfume raw material having a vapor pressure of greater than 0.08 Torr at 25° C.; from about 35% to about 65%, by total weight, of a perfume raw material having a vapor pressure from 0.006 Torr at 25° C. to 0.08 Torr at 25° C.; and from about 20% to about 30%, by total weight, of a perfume raw material having a vapor pressure of less than 0.006 Torr at 25° C.

The fragrance accord of the wet wipe may be neat. The lotion composition of the wet wipe may be aqueous. The lotion composition may comprise an emollient. The emollient may comprise an oil material. The substrate may comprise a non-woven material. The substrate of the wet wipe may comprise a co-form structure. The fragrance accord may comprise from about 10% to about 15%, by total weight, of a perfume raw material having a Kovats Index of less than 1300; from about 35% to about 65%, by total weight, of a perfume raw material having a Kovats Index from 1300 to 1450; and from about 20% to about 30%, by total weight, of a perfume raw material having a Kovats Index of greater than 1450.

Aspects of the present disclosure include a wet wipe comprising a substrate and a lotion composition, wherein the lotion composition comprises a fragrance accord. The fragrance accord comprising: from about 10% to about 20%, by total weight, of a perfume raw material having a vapor pressure of greater than 0.08 Torr at 25° C.; from about 20% to about 30%, by total weight, of a perfume raw material having a vapor pressure from 0.006 Torr at 25° C. to 0.08 Torr at 25° C.; and from about 45% to about 70%, by total weight, of a perfume raw material having a vapor pressure of less than 0.006 Torr at 25° C.

### BRIEF DESCRIPTION OF THE DRAWINGS

FIG. 1 is a schematic, perspective view of an exemplary substrate.

FIG. 2 is a cross-sectional view of the substrate of FIG. 1 taken along line 2-2.

FIG. 3 is a schematic, perspective partially cut-away view of an exemplary substrate.

FIG. 4 is a schematic, cross-sectional view of an exemplary substrate.

FIG. 5 is a schematic, plan view of an exemplary absorbent article in the form of a diaper.

### DETAILED DESCRIPTION

The following definitions may be useful in understanding the present disclosure:

“Soil” refers herein to matter that is extraneous to a surface being cleaned. For example, soils include body exudates, household matter, and outdoor matter. Body exudates include feces, menses, urine, vomitus, mucus, and the like. Household matter includes food, beverages, combinations thereof, and the like. Outdoor matter includes dirt, mud, snow, paint, crayons, and the like.

“Substrate” refers herein to a material which is primarily two-dimensional (i.e., in an XY plane) and whose thickness (in a Z direction) is relatively small (i.e., 1/10 or less) in comparison to the substrate's length (in an X direction) and width (in a Y direction). Non-limiting examples of substrates include a web, layer or layers or fibrous materials, nonwovens, films and foils such as polymeric films or metallic foils. These materials may be used alone or may comprise two or more layers joined together. As such, a web is a substrate.

“Nonwoven” refers herein to a fibrous structure made from an assembly of continuous fibers, coextruded fibers, non-continuous fibers and combinations thereof, without weaving or knitting, by processes such as spunbonding, carding, meltblowing, airlaying, wetlaying, coforming, or other such processes known in the art for such purposes.

“Fiber” and/or “Filament” as used herein means an elongate particulate having an apparent length greatly exceeding its apparent width, i.e., a length to diameter ratio of at least about 10. For purposes of the present invention, a “fiber” is an elongate particulate as described above that exhibits a length of less than 5.08 cm (2 in.) and a “filament” is an elongate particulate as described above that exhibits a length of greater than or equal to 5.08 cm (2 in.).

“Loading” refers to a process of applying a lotion composition to a substrate to form a wet wipe. A “loaded” substrate is associated with a lotion composition.

As used herein, percentages are given as the weight of the component to the total weight of the lotion composition, unless otherwise indicated. Percentages reflect 100% active component material. For example, if a component is available in a dispersion at a concentration of 50% component to dispersion, by total weight, twice as much of the dispersion, by total weight, would be added to the lotion composition to provide the equivalent of 100% active component.

Values disclosed herein as ends of ranges are not to be understood as being strictly limited to the exact numerical values recited. Instead, unless otherwise specified, each numerical range is intended to mean both the recited values and any integers within the range. For example a range disclosed as “1 to 10” is intended to mean “1, 2, 3, 4, 5, 6, 7, 8, 9, and 10”.

“Fragrance accord” refers to a mixture of two or more fragrance raw materials.

“Neat” refers to a fragrance accord that is free from extraneous matter. A neat fragrance accord comprises only perfume raw materials and is unencapsulated and/or unbound from other compounds that would cause a delay in the release of the perfume raw materials of the fragrance accord.

“Aqueous” refers to a lotion composition comprising water as a solvent.

“Perfume raw material” refers to a single individual chemical compound that is odiferous.

The present disclosure includes absorbent articles comprising fragrance accords. In particular, the present disclosure includes absorbent articles having different fragrance accords to meet the needs of various types of consumers.

A consumer may have multiple scent experiences during a diaper change. For example, one scent experience may occur as the consumer opens the package of wipes and removes a wipe from the package. Another scent experience may occur as the consumer cleans a baby's skin to remove soils such as urine or feces. In addition, another scent experience may occur as a result of scent left on the skin of the baby and/or the consumer after cleaning the baby's skin.

It has been found that different consumers may prefer different scents or amount of scent during each scent experience. For example, one type of consumer may prefer a noticeable amount of scent during each scent experience. For example, the same type of consumer may want enough scent present on the absorbent article such that the scent is noticeable as the absorbent article is removed from the package and such that the scent masks the smell of a soil during the cleaning process. The same type of consumer may want a lingering scent left on the skin of the baby and/or the consumer after using the absorbent article. Another type of consumer may want a lower level of scent present on an absorbent article than the type of consumer previously described. For example, the same type of consumer may want a relatively low amount of scent present as the consumer removes the absorbent article from the package. The same type of consumer may want just enough scent to be detectable, without the scent being overwhelming as the absorbent article is removed from the package. During the cleaning process, the same type of consumer may want to experience a noticeable amount of scent that signals that the baby's skin is clean and fresh, but again without the scent from the absorbent article overwhelming the experience. The same type of consumer may want little to no lingering scent left on the baby's and/or the consumer's skin after use of the absorbent article. Yet another type of consumer may want no scent at all.

In order to meet the needs of various types of consumers, the present disclosure includes an array of absorbent articles comprising different fragrance accords. The array of absorbent articles may include a first absorbent article having a first fragrance accord and a second absorbent article having a second fragrance accord. The first fragrance accord may be different from the second fragrance accord. The absorbent articles may include diapers and/or wipes. Wipes of the present disclosure may include a lotion composition comprising a fragrance accord of the present disclosure. While the present disclosure discusses fragrance accords in combination with wipes and diapers, it is to be appreciated that the fragrance accord of the present disclosure may be used with various types of absorbent articles, including diapers, wipes, tissues, feminine hygiene products, adult incontinence products, and cleaning articles, such as disposable dusters.

Absorbent articles in an array may be sold under the same brand and/or trade name.

### Fragrance Accord

Absorbent articles of the present disclosure may include a fragrance accord. The fragrance accord may comprise a perfume raw material or a mixture of different perfume raw materials. Perfume raw materials may be characterized by vapor pressure (VP) or Kovats Index (KI). As used herein, “vapor pressure” refers to an estimated vapor pressure provided by EPI Suite, version 4.0, available from U.S. Environmental Protection Agency.

Perfume raw materials of the present disclosure may be classified as top notes, middle notes, or base notes based upon the relative volatility of a particular perfume raw material. Perfume raw materials having a vapor pressure of greater than 0.08 Torr at 25° C. or a Kovats Index of less than 1300 are classified as “top notes”. Perfume raw materials having a vapor pressure between 0.006 Torr and 0.08 Torr at 25° C. or a Kovats Index between 1300 and 1450 are classified as “middle notes”. Perfume raw materials having a vapor pressure less than or equal to 0.006 Torr at 25° C. or a Kovats Index of greater than 1450 are classified as “base notes”. Top notes are the most volatile and, therefore, provide an initial “burst” of fragrance. Middle notes are less volatile than top notes and may be detected for a longer period of time than top notes. Base notes are the least volatile of top, middle, and base notes and may be detected for the longest period of time.

The present disclosure includes different fragrance accords to meet the needs of various types of consumers. In some exemplary configurations, in order to meet the needs of a consumer wanting a low level of scent present as an absorbent article is removed from a package and during use of the absorbent article, and little to no scent left on the skin of the consumer and/or the baby after use of the absorbent article, an absorbent article may comprise a perfume accord comprising a higher percentage of middle notes as compared with the percentages of top and base notes. For example, an absorbent article may include a fragrance accord comprising from about 10% to about 15%, by total weight, of a perfume raw material having a vapor pressure of greater than 0.08 Torr at 25° C.; from about 35% to about 65%, by total weight, of a perfume raw material having a vapor pressure from 0.006 Torr at 25° C. to 0.08 Torr at 25° C.; and from about 20% to about 30%, by total weight, of a perfume raw material having a vapor pressure of less than 0.006 Torr at 25° C. In another example, an absorbent article may include a fragrance accord comprising from about 10% to about 15%, by total weight, of a perfume raw material having a Kovats Index of less than 1300; from about 35% to about 65%, by total weight, of a perfume raw material having a Kovats Index from 1300 to 1450; and from about 20% to about 30%, by total weight, of a perfume raw material having a Kovats Index of greater than 1450. Without wishing to be bound by theory, it is believed that a perfume accord having a disproportionate amount of middle notes than top or base notes may provide one type of consumer with enough scent as the absorbent article is removed from the package without being overwhelming, while providing enough scent during use of the absorbent article to be detectable by the consumer, and while minimizing the amount of scent that will be left on the baby's and/or the consumer's skin after use of the absorbent article.

In another exemplary configuration, in order to meet the needs of a consumer wanting a relatively high level of scent present as an absorbent article is removed from a package, during use, and left on the skin of the consumer's and/or the baby's skin after use of the absorbent article, an absorbent article may include a perfume accord comprising the highest percentage of base notes and the lowest percentage of top notes. For example, an absorbent article may include a fragrance accord comprising from about 10% to about 20%, by total weight, of a perfume raw material having a vapor pressure of greater than 0.08 Ton at 25° C.; from about 20% to about 30%, by total weight, of a perfume raw material having a vapor pressure from about 0.006 Torr at 25° C. to 0.08 Torr at 25° C.; and from about 45% to about 70%, by total weight, of a perfume raw material having a vapor pressure of less than 0.006 Torr at 25° C. In another example, an absorbent article may comprise from about 10% to about 20%, by total weight, of a perfume raw material having a Kovats Index of less than 1300; from about 20% to about 30%, by total weight, of a perfume raw material having a Kovats Index from 1300 to 1450; and from about 45% to about 70%, by total weight, of a perfume raw material having a Kovats Index of greater than 1450. Without wishing to be bound by theory, it is believed that a fragrance accord having the lowest percent of top notes, a higher percentage of middle notes, and the highest percentage of base notes may provide one type of consumer with a relatively high amount of scent as the absorbent article is removed from the package, with an increase in scent as the absorbent article is being used in order to mask the scent of the soil being removed, and a long lasting scent that remains on the skin of the baby and/or the consumer after using the absorbent article.

In yet another exemplary configuration, in order to meet the needs of a consumer wanting a relatively high level of scent present as an absorbent article is removed from a package, during use, and left on the skin of the baby and/or the consumer after use of the absorbent article, an absorbent article may include a perfume accord comprising a relatively high percentage of top and base notes and the lowest percentage of middle notes as compared to the top and base notes. For example, an absorbent article may include a fragrance accord comprising from about 45% to about 70%, by total weight, of a perfume raw material having a vapor pressure of greater than 0.08 Torr at 25° C.; from about 10% to about 25%, by total weight, of a perfume raw material having a vapor pressure from 0.006 Torr at 25° C. to 0.08 Torr at 25° C.; and from about 20% to about 30%, by total weight, of a perfume raw material having a vapor pressure of less than 0.006 Torr at 25° C. In another example, an absorbent article may include a fragrance accord comprising from about 45% to about 70%, by total weight, of a perfume raw material having a Kovats Index of less than 1300; from about 10% to about 25%, by total weight, of a perfume raw material having a Kovats Index from 1300 to 1450; and from about 20% to about 30%, by total weight, of a perfume raw material having a Kovats Index of greater than 1450. Without wishing to be bound by theory, it is believed that a fragrance accord having a high percentage of top notes and base notes and the lowest percentage of middle notes may provide one type of consumer with a relatively high amount of scent as the absorbent article is removed from the package, with a high level of scent present as the absorbent article is being used in order to mask the scent of the soil being removed, and a long lasting scent that remains on the skin of the baby and/or the consumer after using the absorbent article.

In some exemplary configurations, an absorbent article may be substantially free of a fragrance accord. Without wishing to be bound by theory, it is believed that an absorbent article that is substantially free of a fragrance accord may satisfy the needs of a consumer wanting an absorbent article that is free of scent during the various scent experiences of an absorbent article.

The present disclosure may include an array of absorbent articles to meet the needs of various types of consumers. The array of absorbent articles may include a first absorbent article having a first fragrance accord. The first fragrance accord may comprise from about 10% to about 15%, by total weight, of a perfume raw material having a vapor pressure of greater than 0.08 Torr at 25° C.; from about 35% to about 65%, by total weight, of a perfume raw material having a vapor pressure from 0.006 Torr at 25° C. to 0.08 Torr at 25° C.; and from about 20% to about 30%, by total weight, of a perfume raw material having a vapor pressure of less than 0.006 Torr at 25° C. The array may comprise a second absorbent article having a second fragrance accord. The second fragrance accord may comprise from about 10% to about 20%, by total weight, of a perfume raw material having a vapor pressure of greater than 0.08 Torr at 25° C.; from about 20% to about 30%, by total weight, of a perfume raw material having a vapor pressure from 0.006 Torr at 25° C. to 0.08 Torr at 25° C.; and from about 45% to about 70%, by total weight, of a perfume raw material having a vapor pressure of less than 0.006 Torr at 25° C.

In some exemplary configurations, the array of absorbent articles may include a third absorbent article having a third fragrance accord. The third fragrance accord may comprise from about 45% to about 70%, by total weight, of a perfume raw material having a vapor pressure of greater than 0.08 Torr at 25° C.; from about 10% to about 25%, by total weight, of a perfume raw material having a vapor pressure from 0.006 Torr at 25° C. to 0.08 Torr at 25° C.; and from about 20% to about 30%, by total weight, of a perfume raw material having a vapor pressure of less than 0.006 Torr at 25° C.

In another example, an array of absorbent articles may include a first absorbent article having a first fragrance accord. The first fragrance accord may comprise from about 10% to about 15%, by total weight, of a perfume raw material having a Kovats Index of less than 1300; from about 35% to about 65%, by total weight, of a perfume raw material having a Kovats Index from 1300 to 1450; and from about 20% to about 30%, by total weight, of a perfume raw material having a Kovats Index of greater than 1450. The array may include a second absorbent article having a second fragrance accord. The second fragrance accord may comprise from about 10% to about 20%, by total weight, of a perfume raw material having a Kovats Index of less than 1300; from about 20% to about 30%, by total weight, of a perfume raw material having a Kovats Index from 1300 to 1450; and from about 45% to about 70%, by total weight, of a perfume raw material having a Kovats Index of greater than 1450.

In some exemplary configurations, an array of absorbent articles may include a third absorbent article having a third fragrance accord. The third fragrance accord may comprise from about 45% to about 70%, by total weight, of a perfume raw material having a Kovats Index of less than 1300; from about 10% to about 25%, by total weight, of a perfume raw material having a Kovats Index from 1300 to 1450; and from about 20% to about 30%, by total weight, of a perfume raw material having a Kovats Index of greater than 1450.

In some exemplary configurations, an array of absorbent articles may include an absorbent article that is substantially free of a fragrance accord.

Exemplary top note perfume raw materials of the present disclosure include Prenyl acetate, Dihydro Myrcenol, Ethyl acetate, cis-3-Hexenal, beta Pinene, Eucalyptol, Benzaldehyde, Phenyl acetaldehyde, Melonal, Orange Terpenes, and Amyl Propionate. It is to be appreciated that various other top note perfume raw materials may be used in the fragrance accord of the present disclosure.

Exemplary middle note perfume raw materials of the present disclosure include Mayol, 4-tertiary Butyl Cyclohexyl Acetate, Patchone, Coumarin, Phenyl Ethyl Acetate, Terpinyl acetate, Cinnamic Alcohol, Nonalactone, Dimethyl Benzyl Carbinyl Acetate, Heliotropin, and Undecavertol. It is to be appreciated that various other middle note perfume raw materials may be used in the fragrance accord of the present disclosure.

Exemplary base note perfume raw materials of the present disclosure include Methyl Cedrylone, Eugenol, Frutene, Helional, Sandalore, Helvetolide, iso E Super, Ethyl Vanillin, Karanal, Amyl Salicylate, and Benzoin. It is to be appreciated that various other base note perfume raw materials may be used in the fragrance accord of the present disclosure.

The fragrance accords of the present disclosure may be “neat”.

The fragrance accords of the present disclosure may include various perfume raw materials having various characters, including light, fresh, fruity, citrus, green, floral, watery, powdery, gourmand, woody, oriental, herbal, marine, ozonic, amber, musk, aldehydic, aromatic, spice, balsamic, sweet, and the like.

### Absorbent Article

The fragrance accords of the present disclosure may be used in various types of absorbent articles, including wipes, diapers, feminine hygiene articles, and adult incontinence articles. Without wishing to be bound by theory, it is believed that the fragrance accords of the present disclosure enhance the experience of various types of consumers in the use of absorbent articles such as diapers and wipes.

Wipe

Various fragrance accords of the present disclosure may be incorporated into an absorbent article such as a wipe. The wipe may comprise a substrate and a lotion composition incorporated into the substrate.

Substrate

The substrate may take the form of a wet or dry wipe. The substrate may be a nonwoven material. The substrate may be homogeneous or may be layered. If layered, the substrate may comprise at least two and/or at least three and/or at least four and/or at least five layers. The nonwoven material may comprise one or more layers of such fibrous assemblies, wherein each layer may include continuous fibers, coextruded fibers, non-continuous fibers and combinations thereof.

The substrate of the present invention may include co-formed structure. “Co-formed structure” as used herein means that the structure comprises a mixture of at least two different materials wherein at least one of the materials comprises a filament, such as a polypropylene filament, and at least one other material, different from the first material, comprises a solid additive, such as a fiber and/or a particulate. In one example, a co-formed fibrous structure comprises solid additives, such as fibers, such as wood pulp fibers and/or absorbent gel materials and/or filler particles and/or particulate spot bonding powders and/or clays, and filaments, such as polypropylene filaments. The substrate may include fibers comprised of any natural, cellulosic, and/or wholly synthetic material. Fibers are typically considered discontinuous in nature. Examples of natural fibers may include cellulosic natural fibers, such as fibers from hardwood sources, softwood sources, or other non-wood plants. The natural fibers may comprise cellulose, starch and combinations thereof. Non-limiting examples of suitable cellulosic natural fibers include wood pulp, typical northern softwood Kraft, typical southern softwood Kraft, typical CTMP, typical deinked, corn pulp, acacia, eucalyptus, aspen, reed pulp, birch, maple, radiata pine and combinations thereof. Other sources of natural fibers from plants include albardine, esparto, wheat, rice, corn, sugar cane, papyrus, jute, reed, sabia, raphia, bamboo, sidal, kenaf, abaca, sunn, rayon (also known as viscose), lyocell, cotton, hemp, flax, ramie, bagasse and combinations thereof. Yet other natural fibers may include fibers from other natural non-plant sources, such as, down, feathers, silk, cotton and combinations thereof. The natural fibers may be treated or otherwise modified mechanically or chemically to provide desired characteristics or may be in a form that is generally similar to the form in which they can be found in nature. Mechanical and/or chemical manipulation of natural fibers does not exclude them from what are considered natural fibers with respect to the development described herein.

The substrate may include synthetic fibers. The synthetic fibers can be any material, such as those selected from the group consisting of polyesters (e.g., polyethylene terephthalate), polyolefins, polypropylenes, polyethylenes, polyethers, polyamides, polyesteramides, polyvinylalcohols, polyhydroxyalkanoates, polysaccharides, and combinations thereof. Further, the synthetic fibers can be a single component (i.e., single synthetic material or mixture makes up entire fiber), bi-component (i.e., the fiber is divided into regions, the regions including two or more different synthetic materials or mixtures thereof and may include co-extruded fibers and core and sheath fibers) and combinations thereof. Bicomponent fibers can be used as a component fiber of the structure, and/or they may be present to act as a binder for the other fibers present in the fibrous structure. Any or all of the synthetic fibers may be treated before, during, or after manufacture to change any desired properties of the fibers. The substrate may comprise hydrophilic fibers, hydrophobic fibers, or a combination thereof.

The substrate may comprise various percentages of natural and/or synthetic fibers. For example, in some exemplary configurations, the substrate may comprise 100% synthetic fibers. In another exemplary configuration, the substrate may comprise natural and synthetic fibers. For example, the substrate may comprise from about 0% to about 90% natural fibers, with the balance comprising synthetic fibers. The substrate may be comprised of 10%, 20%, 30%, 40%, 50%, 60%, 70%, or 80% natural fibers.

The substrate may include filaments. Filaments are typically considered continuous or substantially continuous in nature. Non-limiting examples of filaments include meltblown and/or spunbond filaments. Non-limiting examples of materials that can be spun into filaments include natural polymers, such as starch, starch derivatives, cellulose and cellulose derivatives, hemicellulose, hemicellulose derivatives, chitin, chitosan, polyisoprene (cis and trans), peptides, polyhydroxyalkanoates, and synthetic polymers including, but not limited to, thermoplastic polymer filaments comprising thermoplastic polymers, such as polyesters, nylons, polyolefins such as polypropylene filaments, polyethylene filaments, polyvinyl alcohol and polyvinyl alcohol derivatives, sodium polyacrylate (absorbent gel material) filaments, and copolymers of polyolefins such as polyethylene-octene, and biodegradable or compostable thermoplastic fibers such as polylactic acid filaments, polyvinyl alcohol filaments, and polycaprolactone filaments. The filaments may be monocomponent or multicomponent, such as bicomponent filaments.

The substrate may comprise a plurality of filaments, a plurality of solid additives, such as fibers, and a mixture of filaments and solid additives.

In certain configurations, it may be desirable to have particular combinations of fibers to provide desired characteristics. For example, it may be desirable to have fibers of certain lengths, widths, coarseness or other characteristics combined in certain layers, or separate from each other. The fibers may be of virtually any size and may have an average length from about 1 mm to about 60 mm. Average fiber length refers to the length of the individual fibers if straightened out. The fibers may have an average fiber width of greater than about 5 micrometers. The fibers may have an average fiber width of from about 5 micrometers to about 50 micrometers. The fibers may have a coarseness of greater than about 5 mg/100 m. The fibers may have a coarseness of from about 5 mg/100 m to about 75 mg/100 m.

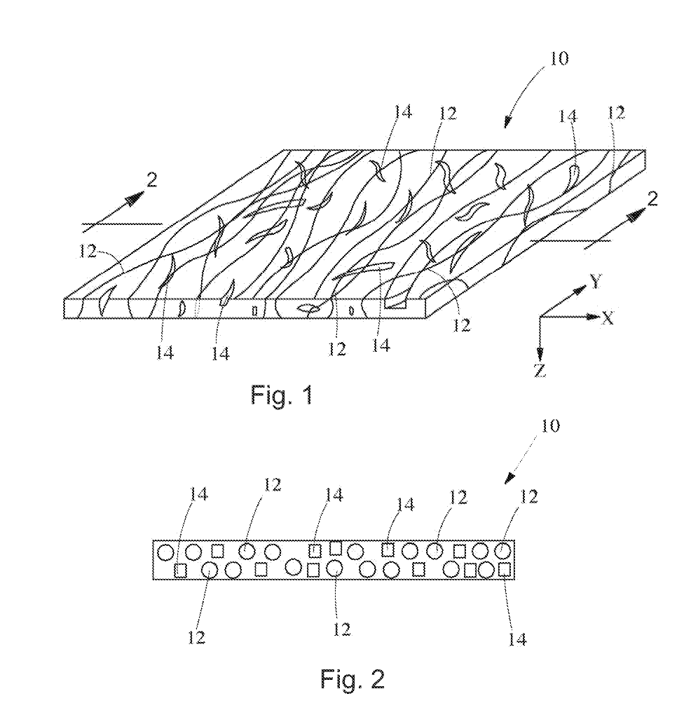

FIGS. 1 and 2 show schematic representations of an example substrate. As shown in FIGS. 1 and 2, the substrate 10 may be a co-formed structure. The substrate 10 comprises a plurality of filaments 12, such as polypropylene filaments, and a plurality of solid additives, such as wood pulp fibers 14. The filaments 12 may be randomly arranged as a result of the process by which they are spun and/or formed into the fibrous structure 10. The wood pulp fibers 14, may be randomly dispersed throughout the fibrous structure 10 in the x-y plane. The wood pulp fibers 14 may be non-randomly dispersed throughout the fibrous structure in the z-direction. In one example (not shown), the wood pulp fibers 14 are present at a higher concentration on one or more of the exterior, x-y plane surfaces than within the fibrous structure along the z-direction.

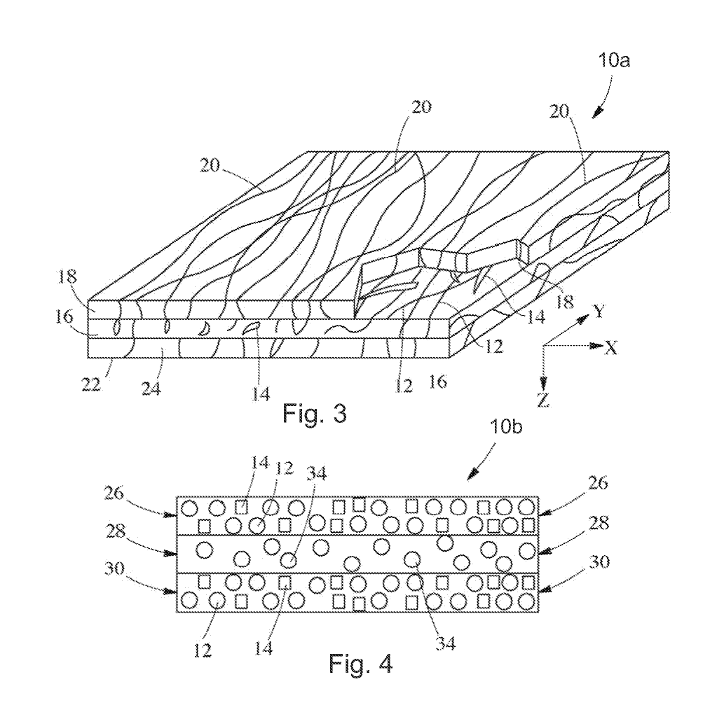

FIG. 3 shows another exemplary configuration of a layered substrate 10a. The layered substrate 10a comprises a first layer 16 comprising a plurality of filaments 12, such as polypropylene filaments, and a plurality of solid additives, in this example, wood pulp fibers 14. The layered substrate 10a further comprises a second layer 18 comprising a plurality of filaments 20, such as polypropylene filaments. In one example, the first and second layers 16, 18, respectively, are sharply defined zones of concentration of the filaments and/or solid additives. The plurality of filaments 20 may be deposited directly onto a surface of the first layer 16 to form a layered substrate 10a that comprises the first and second layers 16, 18, respectively.

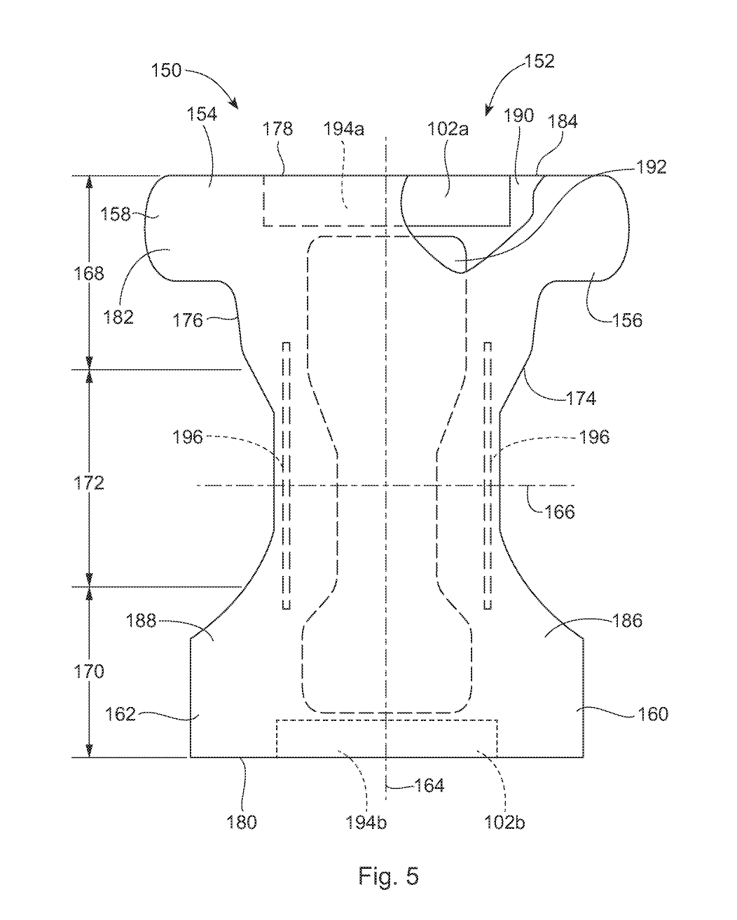

Further, the layered substrate 10a may comprise a third layer 22, as shown in FIG. 5. The third layer 22 may comprise a plurality of filaments 24, which may be the same or different from the filaments 20 and/or 16 in the second 18 and/or first 16 layers. As a result of the addition of the third layer 22, the first layer 16 is positioned, for example sandwiched, between the second layer 18 and the third layer 22. The plurality of filaments 24 may be deposited directly onto a surface of the first layer 16, opposite from the second layer 18, to form the layered substrate 10a that comprises the first, second and third layers 16, 18, 22, respectively.

FIG. 4 shows another exemplary substrate in the form of a layered substrate 10b. The layered substrate 10b comprises a first layer 26, a second layer 28 and optionally a third layer 30. The first layer 26 comprises a plurality of filaments 12, such as polypropylene filaments, and a plurality of solid additives, such as wood pulp fibers 14. The second layer 28 may comprise any suitable filaments, solid additives and/or polymeric films. In one example, the second layer 28 comprises a plurality of filaments 34. In one example, the filaments 34 comprise a polymer selected from the group consisting of: polysaccharides, polysaccharide derivatives, polyvinylalcohol, polyvinylalcohol derivatives and mixtures thereof.

In other exemplary configurations, a substrate may comprise two outer layers consisting of 100%, by total weight, filaments and an inner layer consisting of 100%, by total weight, fibers.

The substrate may comprise any suitable amount of filaments and any suitable amount of solid additives. For example, the substrate may comprise from about 10% to about 70% and/or from about 20% to about 60% and/or from about 30% to about 50% by dry weight of the substrate of filaments and from about 90% to about 30% and/or from about 80% to about 40% and/or from about 70% to about 50% by dry weight of the substrate of solid additives, such as wood pulp fibers. In one example, the substrate comprises filaments.

The filaments and solid additives may be present in substrate according to the present invention at weight ratios of filaments to solid additives of from at least about 1:1 and/or at least about 1:1.5 and/or at least about 1:2 and/or at least about 1:2.5 and/or at least about 1:3 and/or at least about 1:4 and/or at least about 1:5 and/or at least about 1:7 and/or at least about 1:10.

Non-limiting examples of suitable polypropylenes for making the filaments of the present invention are commercially available from Lyondell-Basell and Exxon-Mobil.

Any hydrophobic or non-hydrophilic materials within the substrate, such as polypropylene filaments, may be surface treated and/or melt treated with a hydrophilic modifier. Non-limiting examples of surface treating hydrophilic modifiers include surfactants, such as Triton X-100. Non-limiting examples of melt treating hydrophilic modifiers that are added to the melt, such as the polypropylene melt, prior to spinning filaments, include hydrophilic modifying melt additives such as VW351 and/or S-1416 commercially available from Polyvel, Inc. and Irgasurf commercially available from Ciba. The hydrophilic modifier may be associated with the hydrophobic or non-hydrophilic material at various levels. In one example, the hydrophilic modifier is associated with the hydrophobic or non-hydrophilic material at a level of less than about 20% and/or less than about 15% and/or less than about 10% and/or less than about 5% and/or less than about 3% to about 0% by dry weight of the hydrophobic or non-hydrophilic material.

The substrate may include optional additives, each, when present, at individual levels of from about 0% and/or from about 0.01% and/or from about 0.1% and/or from about 1% and/or from about 2% to about 95% and/or to about 80% and/or to about 50% and/or to about 30% and/or to about 20% by dry weight of the substrate. Non-limiting examples of optional additives include permanent wet strength agents, temporary wet strength agents, dry strength agents such as carboxymethylcellulose and/or starch, softening agents, lint reducing agents, opacity increasing agents, wetting agents, odor absorbing agents, temperature indicating agents, color agents, dyes, osmotic materials, microbial growth detection agents, antibacterial agents and mixtures thereof.

The fibers may be circular in cross-section, dog-bone shape, delta (i.e., triangular cross section), trilobal, ribbon, or other shapes typically produced as staple fibers. Likewise, the fibers can be conjugate fibers such as bicomponent fibers. The fibers may be crimped and may have a finish, such as a lubricant, applied.

The substrate materials may also be treated to improve the softness and texture thereof. The substrate may be subjected to various treatments, such as physical treatment, hydro-molding, hydro-embossing, and ring rolling, as described in U.S. Pat. No. 5,143,679; structural elongation, as described in U.S. Pat. No. 5,518,801; consolidation, as described in U.S. Pat. Nos. 5,914,084; 6,114,263; 6,129,801 and 6,383,431; stretch aperturing, as described in U.S. Pat. Nos. 5,628,097; 5,658,639; and 5,916,661; differential elongation, as described in U.S. Pat. No. 7,037,569, and other solid state formation technologies as described in U.S. Pat. No. 7,553,532 and U.S. Pat. No. 7,410,683; zone activation, and the like; chemical treatment, such as rendering part or all of the substrate hydrophobic, and/or hydrophilic, and the like; thermal treatment, such as thermal-embossing, softening of fibers by heating, thermal bonding and the like; and combinations thereof.

Without wishing to be bound by theory, it is believed that a textured substrate may further enable the ease of removal of soils by improving the ability to grip or otherwise lift the soils from the surface during cleansing. Any one of a number of texture elements may be useful in improving the ability to grip or otherwise lift the soil from the surface during cleansing such as continuous hydro-molded elements, hollow molded element, solid molded elements, circles, squares, rectangles, ovals, ellipses, irregular circles, swirls, curly cues, cross hatches, pebbles, lined circles, linked irregular circles, half circles, wavy lines, bubble lines, puzzles, leaves, outlined leaves, plates, connected circles, changing curves, dots, honeycombs, and the like, and combinations thereof. The texture elements may be hollow elements. The texture elements may be connected to each other. The texture elements may overlap each other.

The substrate may have a basis weight between about 15, 30, 40, or 45 grams/m² and about 65, 75, 85, 95, or 100 grams/m². A suitable substrate may be a carded nonwoven comprising a 40/60 blend of viscose fibers and polypropylene fibers having a basis weight of 58 grams/m² as available from Suominen of Tampere, Finland as FIBRELLA® 3160. FIBRELLA® 3160 is a 58 grams/m² nonwoven web comprising 60%, by total weight, of 1.5 denier polypropylene fibers and 40%, by total weight, of 1.5 denier viscose fibers. Another suitable material may be FIBRELLA® 3100 which is a 62 grams/m² nonwoven web comprising 50%, by total weight, of 1.5 denier polypropylene fibers and 50%, by total weight, of 1.5 denier viscose fibers. In both of these commercially available fibrous webs, the average fiber length is about 38 mm. Another suitable material for use as a substrate may be SAWATEX® 2642 as available from Sandler AG of Schwarzenbach/Salle, Germany. Yet another suitable material for use as a substrate may have a basis weight of from about 50 grams/m² to about 60 grams/m² and have a 20/80 blend of viscose fibers and polypropylene fibers. The substrate may also be a 60/40 blend of pulp and viscose fibers. Exemplary nonwoven substrates are described in U.S. Patent Publication 2012/066852 and U.S. Patent Publication U.S. 2011/244199.

In some configurations, the surface of the substrate may be essentially flat. In other configurations, the surface of the substrate may optionally contain raised and/or lowered portions. The raised and/or lowered portions can be in the form of logos, indicia, trademarks, geometric patterns, and/or images of the surfaces that the substrate is intended to clean (i.e., infant's body, face, etc.). The raised and/or lowered portions may be randomly arranged on the surface of the substrate or be in a repetitive pattern of some form.

In some exemplary configurations, the substrate may be biodegradable. For example, the substrate could be made from a biodegradable material such as a polyesteramide, or a high wet strength cellulose. In some exemplary configurations, the substrate may be dispersible.

Lotion Composition

A wipe may include a lotion composition comprising various fragrance accords of the present disclosure. The lotion composition may be aqueous or emulsion-based. The pH of the composition may be from about pH 3, 4, or 5 to about pH 7, 7.5, or 8. In some exemplary configurations, the pH may be from about 3.5 to about 4.1.

In some exemplary configurations, the lotion composition may comprise a glucomannan. Without being bound by theory, it is believed that a cleansing composition comprising a glucomannan improves the cleaning performance of a wet wipe. Without wishing to be bound by theory, using a cleansing composition comprising a glucomannan in a wet wipe may increase the adhesive interaction between the soil and the wet wipe above the adhesive interaction between the soil and the surface, thereby allowing the soil to detach from the surface upon wiping. The lotion composition may comprise from about 0.01%, by total weight, to about 0.50%, by total weight, of a glucomannan. The lotion composition may include a glucomannan and one or more synergy enhancing agents. Non-limiting examples of synergy enhancing agents include xanthan gum, carrageenan, alginate, locust bean gum, starch, and gellan gum.

The lotion composition may comprise from about 0.1%, by total weight, to about 0.5%, by total weight, or from about 0.12%, by total weight, to about 0.18%, by total weight, of one or more synergy enhancing agents. The ratio of glucomannan to synergy enhancing agent present in the lotion composition may be from about 1:1.5 to about 1:10. Exemplary compositions comprising glucomannan and a synergy enhancing agent are described in U.S. Provisional Patent Application No. 61/758,802.

An exemplary wet wipe may include a lotion composition comprising glucomannan and xanthan gum. Another exemplary wet wipe may include a lotion composition comprising glucomannan, carrageenan, and xanthan gum. In a lotion composition comprising glucomannan, carrageenan, and xanthan gum, the ratio of xanthan gum to glucomannan to carrageenan may be from about 1:0.02:0.03 to about 1:0.33:0.5.

The peak complex viscosity of a lotion composition comprising a glucomannan and a synergy enhancing agent for use in a wet wipe may be greater than about 0.8 Pascal-seconds (hereinafter “Pa·s”), greater than 2.5 Pa·s, or greater than about 3.0 Pa·s. The peak complex viscosity of a lotion composition for use in a wet wipe may be in the range of about 1.0 Pa·s to about 5.0 Pa·s.

In addition, the lotion composition may include various optional ingredients, such as surfactants, emollients, film-formers, preservatives, pH buffers, rheology modifiers, and various other adjunct ingredients, such as described in U.S. Pat. Nos. 7,666,827; 7,005,557; 8,221,774; and U.S. Patent Application Publication No. 2011/0268777. It is to be noted that some ingredient compounds can have a multiple function and that all compounds are not necessarily present in the lotion composition.

Emollient

The lotion composition may include an emollient. Emollients may (1) hydrate the residues (for example, fecal residues or dried urine residues or menses), thus enhancing their removal from the skin, (2) hydrate the skin, thus reducing its dryness and irritation while improving its flexibility under the wiping movement, (3) reduce the adhesive interaction between the soil and the surface, and (4) protect the skin from later irritation (for example, caused by the friction of an absorbent article) as the emollient is deposited onto the skin and remains at its surface as a thin protective layer.

An emollient may include silicone oils, functionalized silicone oils, hydrocarbon oils, fatty alcohols, fatty alcohol ethers, fatty acids, esters of monobasic and/or dibasic and/or tribasic and/or polybasic carboxylic acids with mono and polyhydric alcohols, polyoxyethylenes, polyoxypropylenes, mixtures of polyoxyethylene and polyoxypropylene ethers of fatty alcohols, and mixtures thereof. The emollients may be either saturated or unsaturated, have an aliphatic character and be straight or branched chained or contain alicyclic or aromatic rings.

In some exemplary configurations, the lotion composition may comprise a mixture of caprylic/capric triglycerides in combination with Bis-PEG/PPG-16/16 PEG/PPG-16/16 dimethicone known as ABIL® CARE 85, available from Degussa Care Specialties of Hopewell, Va.

Various oil materials may function as emollients, while also providing skin benefits, including improving or maintain the integrity of the skin. For example, the lotion composition may comprise an omega-6 fatty acid. The lotion composition may comprise at least about 0.003%, from about 0.003% to about 35%, from about 0.015% to about 25%, or from about 0.06% to about 20%, by total weight, of the lotion and/or coating composition, of omega-6 fatty acid. Exemplary lotion compositions comprising omega-6 fatty acids are described in U.S. Patent Publication No. 2011/0159074 A1.

The omega-6 fatty acid may be added to the lotion composition as an oil material, such as from a vegetable oil. Therefore, in one exemplary configuration, the lotion composition comprises an oil material comprising omega-6 fatty acid. The lotion composition may comprise from about 0.1% to about 70%, from about 0.5% to about 50%, or from about 2% to about 40%, by total weight, of the lotion and/or coating composition, of the oil material. The oil material may comprise at least 3%, from about 3% to about 50%, or from about 5% to about 40%, by total weight, of the oil material, of omega-6 fatty acid.

Non-limiting examples of suitable oil materials include high oleic canola Oil (Brassica campestris, B. napus, B. rapa; characterized by having an oleic fatty acid content greater than 70%, e.g., high oleic canola oil, very high oleic canola oil, or partially hydrogenated canola oil), marula kernel oil (Sclerocarya birrea), palm oil (Elaeis Guineensis Oil), palm olein, palm stearin, palm superolein, pecan oil, pumpkin seed oil, oleic safflower oil (Carthamus Tinctorius; characterized by having an oleic fatty acid content of greater than about 30% and omega-6 fatty acid content of less than about 50%, e.g., high oleic safflower oil), sesame oil (Sesamum indicum, S. oreintale), soybean oil (Glycine max, e.g., high oleic soybean, low linolenic soybean oil, partially hydrogenated), high oleic sunflower oil (Helianthus annus; characterized by having an oleic content of greater than about 40%, e.g., mid oleic sunflower or high oleic sunflower oil), and mixtures thereof. Oleic canola oil, palm oil, sesame oil, high oleic safflower oil, high oleic soybean oil, mid oleic sunflower oil, and high oleic sunflower oil are common plant-bred derived oils and may be also be derived from non-genetically modified organisms (non-GMO).

Non-limiting examples of oil materials are commercially-available from a number of vendors, including Cargill for partially hydrogenated soybean oil (i.e., Preference® 110W Soybean Oil or Preference® 300 Hi Stability Soybean Oil), mid oleic sunflower oil (i.e., NuSun® Mid-Oleic Sunflower Oil), high oleic sunflower oil (i.e., Clear Valley® High Oleic Sunflower Oil), high oleic canola oil, very high oleic canola, and partially hydrogenated low erucic rapeseed oil (i.e., Clear Valley® 65 High Oleic Canola Oil and Clear Valley® 75 High Oleic Canola Oil); Lambert Technology for high oleic canola oil (i.e., Oleocal C104); Arch Personal Care for marula kernel oil; Pioneer for high oleic soybean oil (i.e., Plenish®); Asoyia for low linolenic soybean oil (i.e., Ultra Low Linolenic Soybean Oil®); and Dipasa, Inc. for refined sesame oil.

The oil material can further comprise a blend of oils, including those described supra, as well as additional oil materials. Suitable additional oil materials can include acai berry oil, almond oil, avocado oil, beech oil, brazil nut oil, camelina sativa oil (family Brassicaceae, e.g., Camelina Sativa, Gold of Pleasure, False Flax, etc.), camellia seed oil, canola oil, carrot seed oil, cashew nut oil, caster oil, cherry kernel oil, chia oil, corn oil, cottonseed oil, hydrogenated cottonseed oil, evening primrose oil, filbert (hazelnut) oil, grapeseed oil, hemp oil, hickory nut oil, jojoba oil, kukui oil, lanolin, olive oil (Olea europaca), macadamia oil, maringa oil, meadowfoam oil, neem oil, palm kernel oil, olive oil, passionflower oil (family Passiflora, Passiflora Incarnata), peanut oil, peach kernel oil, pistachio nut oil, rapeseed oil, rice bran oil, rose hip oil, safflower oil, sorghum oil, soybean oil, sunflower seed oil, tall oil, vegetable oil, vegetable squalene, walnut oil, wheat germ oil, and mixtures thereof. The oil material of the present invention can be selected from the group consisting of camelina sativa seed oil, oleic canola oil, evening primrose oil, marula kernel oil, palm oil, palm olein, palm stearin, palm superolein, passiflora incarnata seed oil, pecan oil, pumpkin seed oil, oleic safflower oil, sesame oil, soybean oil, oleic sunflower oil, vegetable oil, and mixtures thereof.

Suitable, commercially available oil materials include a mixture of vegetable oil and camelina sativa seed oil (commercially-available as Lipex® Omega 3/6 from Aarhus Karlshamn Sweden AB), a mixture of vegetable oil and passiflora incarnata seed oil (commercially-available as Lipex® Omega Passiflora from Aarhus Karlshamn Sweden AB), a mixture of vegetable oil and evening primrose oil (commercially-available as Lipex Omega EPO from Aarhus Karlshamn Sweden AB), high oleic canola oil (commercially-available as Clear Valley® 75 High Oleic Canola Oil from Cargill), and mixtures thereof.

Surfactant

The lotion composition may include one or more surfactants. The surfactant can be an individual surfactant or a mixture of surfactants. The surfactant may be a polymeric surfactant or a non-polymeric one. The surfactant may aid in dissolution and removal of the soils from the surface being cleansed. The surfactant or combinations of surfactants may be mild, which means that the surfactants provide sufficient cleaning or detersive benefits but do not overly dry or otherwise harm or damage the skin. The surfactant, when present in the lotion composition, may be present in an amount ranging from about 0.5%, 1%, or 4%, by total weight, to about 0.001%, 0.01% or 0.02%, by total weight, of the lotion composition. The surfactant may comprise PEG-40 Hydrogenated Castor Oil, manufactured by Clariant International Ltd. of Switzerland under the designation EMULSOGEN® HCW049.

A wide variety of surfactants are useful herein and include those selected from the group consisting of anionic surfactants, nonionic surfactants, cationic surfactants, amphoteric surfactants, zwitterionic surfactants, and mixtures thereof.

A wide variety of anionic surfactants are useful herein. Non-limiting examples of anionic surfactants include those selected from the group consisting of carboxylates, sarcosinates, sulfates, sulfonates, isethionates, taurates, phosphates, lactylates, glutamates, and mixtures thereof.

Nonionic surfactants useful herein include, but are not limited to, those selected from the group consisting of alkyl glucosides, alkyl polyglucosides, polyhydroxy fatty acid amides, alkoxylated fatty acid esters, alkoxylated fatty alcohol ethers, sucrose esters, and mixtures thereof.

Amphoteric surfactants suitable for use in the present compositions include those surfactants broadly described as derivatives of aliphatic secondary and tertiary amines in which the aliphatic radical can be straight or branched chain and wherein one of the aliphatic substituents contains from about 8 to about 18 carbon atoms and one contains an anionic water solubilizing group such as carboxy, sulfonate, sulfate, phosphate, or phosphonate. Useful amphoteric surfactants include the group consisting of cocoamphoacetate, cocoamphodiacetate, lauroamphoacetate, lauroamphodiacetate, and mixtures thereof.

Zwitterionic surfactants suitable for use herein include those surfactants broadly described as derivatives of aliphatic quaternary ammonium, phosphonium, and sulfonium compounds, in which the aliphatic radicals can be straight or branched chain, and wherein one of the aliphatic substituents contains from about 8 to about 18 carbon atoms and one contains an anionic group such as carboxy, sulfonate, sulfate, phosphate or phosphonate. Useful zwitterionic surfactants include betaines, amphoacetates and sulfobetaines, e.g., cocoamidopropylbetaine, sodium laurylamphoacetate and cocoamidopropylhydroxysultaine.

Rheology Modifier

The cleaning composition may comprise one or more theology modifiers. A rheology modifier may (1) help to stabilize the lotion composition on a substrate, (2) enhance the transfer of the lotion composition to the skin, and (3) enhance the uniformity of the layer of the lotion composition on the skin. For example, rheology modifiers may help to preserve a homogeneous distribution of the lotion composition within a stack of the substrates. Any composition that is in fluid form may have a tendency to migrate to the lower part of the wipes stack during prolonged storage. This effect may create an upper part of the stack of substrates having less lotion composition than the bottom part of the stack.

Non-limiting examples of rheology modifiers include, but are not limited to, rheology modifiers comprising: polysaccharide units, e.g., cellulose, xanthan gum, diutan gum, carrageenan, gellan gum, welan gum, pectin, sclerotium gum, starch, galactoarabinan, alginate, and modified-forms thereof; homopolymers of acrylic acid; acrylic acid cross-linked with a polyfunctional compound, e.g., carbomer and acrylate crosspolymer; copolymers of acrylic acid, acrylate esters, maleic acid and the like, generally known as the alkali swellable emulsions (ASE) group; hydrophobically-modified copolymers of acrylic acid, acrylate esters, maleic acid and the like, generally known as the hydrophobically-modified alkali swellable emulsions (HASE) group; polyethylene glycol units of varying length connected by urethane linkages and terminated with hydrophobic end groups, generally known as the hydrophobically-modified ethoxylated urethane resins (HEUR) group; organoclays; silicas; and combinations thereof.

Rheology modifiers, when present in the lotion composition, may be present in the range of about 0.01%, 0.015%, or 0.02%, by total weight, to about 2%, by total weight, of the lotion composition.

Preservative

Controlling microbiological growth may be beneficial in water based products such as lotion compositions intended for use in wet wipes. The lotion composition may comprise a preservative or a combination of preservatives acting together as a preservative system. Preservatives and preservative systems are used interchangeably in the present disclosure to indicate one unique or a combination of preservative compounds. A preservative may be understood to be a chemical or natural compound or a combination of compounds reducing the growth of microorganisms, thus enabling a longer shelf life for a package of substrates (opened or not opened) as well as creating an environment with reduced growth of microorganisms when transferred to the skin during the wiping process.

The spectrum of activity of the preservative may include bacteria, molds and yeast. Each of such microorganisms may be killed by the preservative. Another mode of action to be contemplated may be the reduction of the growth rate of the microorganisms without active killing. Both actions however result in a drastic reduction of the population of microorganisms.

Materials useful as preservatives include methylol compounds, iodopropynyl compounds, simple aromatic alcohols, paraben compounds, benzyl alcohol, benzoic acid, benzoates, sorbic acid, sorbates, phenoxyethanol, ethxylhexyglycerin, chelators such as ethylenediamine tetraacetic acid, and combinations thereof. Suitable preservative systems are described in U.S. Patent Publication No. 2005/0008680 and U.S. Patent Publication No. 2005/0008681.

Low pH buffering systems, such as a citrate-citric acid buffering system at a pH of less than about 5, may also be employed as part of the preservative system.

In some exemplary configurations, the preservative system may comprise simple aromatic alcohols (e.g., benzyl alcohol). Materials of this type may have effective antibacterial activity. Benzyl alcohol is available from Symrise, Inc. of Teterboro, N.J. In other exemplary configurations, the preservative system may comprise a mixture of benzyl alcohol, sodium benzoate, phenoxyethanol, ethylhexylglycerin, ethylenediamine tetraacetic acid, citric acid, and sodium citrate dehydrate wherein the pH of the lotion composition is less than about 4. The total concentration of benzyl alcohol may be lower than about 0.4%, by total weight, of the lotion composition. The total concentration of sodium benzoate may be lower than about 0.3%, by total weight, of the lotion composition. The combination of phenoxyethanol and ethylhexylglycerin, which are available as EUXYL® PE 9010 from Schulke & Mayr GmbH of Germany, may be lower than about 0.4%.

In some exemplary configurations, acidic compounds used in sufficient amount to reduce the pH of the lotion composition (e.g., pH of less than about 5) may be useful as the preservative, or as a potentiator for other preservative ingredients.

In other exemplary configurations, chelators, such as ethylenediamine tetraacetic acid and its salts, may also be used in preservative systems as a potentiator for other preservative ingredients.

Adjunct Ingredients

The lotion composition may optionally include other adjunct ingredients. Possible adjunct ingredients may be selected from a wide range of additional ingredients such as texturizers, colorants, soothing agents and medically active ingredients, such as healing actives and skin protectants.

Diaper

The fragrance accord of the present disclosure may be incorporated into various other absorbent articles, including diapers. The fragrance accord may be applied to various components of an absorbent article such as a diaper. Various components of the diaper may comprise various fragrance accords of the present disclosure. It is to be appreciated that the fragrance accords may be applied to an absorbent article such as a diaper in various ways. The fragrance accord may be incorporated with a carrier material such as water before applying the fragrance accord to the absorbent article.

For the purposes of a specific illustration, FIG. 5 shows one example of a disposable absorbent article 150 in the form of a diaper 152 that may include the fragrance accord of the present disclosure. In particular, FIG. 5 is a plan view of one embodiment of a diaper 152 including a chassis 154 shown in a flat, unfolded condition, with the portion of the diaper 152 that faces a wearer oriented towards the viewer. A portion of the chassis structure is cut-away in FIG. 5 to more clearly show the construction of and various features that may be included in exemplary configurations of the diaper.

As shown in FIG. 5, the diaper 152 includes a chassis 154 having a first ear 156, a second ear 158, a third ear 160, and a fourth ear 162. To provide a frame of reference for the present discussion, the chassis is shown with a longitudinal axis 164 and a lateral axis 166. The chassis 154 is shown as having a first waist region 168, a second waist region 170, and a crotch region 172 disposed intermediate the first and second waist regions. The periphery of the diaper is defined by a pair of longitudinally extending side edges 174, 176; a first outer edge 178 extending laterally adjacent the first waist region 168; and a second outer edge 180 extending laterally adjacent the second waist region 170. As shown in FIG. 5, the chassis 154 includes an inner, body-facing surface 182, and an outer, garment-facing surface 184.

As shown in FIG. 5, the chassis 154 of the diaper 152 may include an outer covering layer 186 including a topsheet 188 and a backsheet 190. An absorbent core 192 may be disposed between a portion of the topsheet 188 and the backsheet 190. As discussed in more detail below, one or more of the regions may be stretchable and may include an elastomeric material or layered elastic substrate as described herein. As such, the diaper 152 may be configured to adapt to a specific wearer's anatomy upon application and to maintain coordination with the wearer's anatomy during wear. It is to be appreciated that the fragrance accord may be incorporated to various features of the diaper 152, including incorporating the fragrance accord into the topsheet, backsheet, or absorbent core, for example.

The chassis 154 of the diaper 152 may include the backsheet 190, shown for example, in FIG. 5. In some embodiments, the backsheet is configured to prevent exudates absorbed and contained within the chassis from soiling articles that may contact the diaper, such as bedsheets and undergarments. Some embodiments of the backsheet may be fluid permeable, while other embodiments may be impervious to liquids (e.g., urine) and comprises a thin plastic film. Some backsheet films may include those manufactured by Tredegar Industries Inc. of Terre Haute, Ind. and sold under the trade names X15306, X10962, and X10964. Other backsheet materials may include breathable materials that permit vapors to escape from the diaper while still preventing exudates from passing through the backsheet. Exemplary breathable materials may include materials such as woven webs, nonwoven webs, composite materials such as film-coated nonwoven webs, and microporous films. Suitable breathable composite materials are described in greater detail in PCT Application No. WO 95/16746, published on Jun. 22, 1995 and U.S. Pat. No. 5,865,823. Other breathable backsheets including nonwoven webs and apertured formed films are described in U.S. Pat. Nos. 5,571,096 and 6,573,423.

The backsheet 190, or any portion thereof, may be stretchable in one or more directions. In one embodiment, the backsheet may comprise a structural elastic-like film (“SELF”) web. Embodiments of SELF webs are more completely described in U.S. Pat. Nos. 5,518,801; 5,723,087; 5,691,035; 5,916,663; and 6,027,483. In some embodiments, the backsheet may comprise elastomeric films, foams, strands, nonwovens, or combinations of these or other suitable materials with nonwovens or synthetic films. Additional embodiments include backsheets that comprise a stretch nonwoven material; an elastomeric film in combination with an extensible nonwoven; an elastomeric nonwoven in combination with an extensible film; and/or combinations thereof. Details on such backsheet embodiments are more completely described in U.S. Publication Nos. US2007/0287348A1; US2007/0287982A1; and US2007/0287983A1. The backsheet 190 may be joined with the topsheet 188, the absorbent core 192, and/or other elements of the diaper 152 in various ways. For example, the backsheet may be connected with a uniform continuous layer of adhesive, a patterned layer of adhesive, or an array of separate lines, spirals, or spots of adhesive. One embodiment utilizes an open pattern network of filaments of adhesive as disclosed in U.S. Pat. No. 4,573,986. Other embodiments utilize several lines of adhesive filaments which are swirled into a spiral pattern, as is illustrated by the apparatus and methods shown in U.S. Pat. Nos. 3,911,173; 4,785,996; and 4,842,666. In some embodiments, the backsheet is connected with heat bonds, pressure bonds, ultrasonic bonds, dynamic mechanical bonds, or any other suitable attachment means or a combination thereof.

The topsheet 188 may be constructed to be compliant, soft feeling, and non-irritating to the wearer's skin. Further, all or at least a portion of the topsheet 188 may be liquid pervious, permitting liquid to readily penetrate therethrough. As such, the topsheet may be manufactured from a wide range of materials, such as porous foams; reticulated foams; apertured nonwovens or plastic films; or woven or nonwoven webs of natural fibers (e.g., wood or cotton fibers), synthetic fibers (e.g., polyester or polypropylene fibers), or a combination of natural and synthetic fibers. One example of a topsheet including a web of staple length polypropylene fibers is manufactured by Veratec, Inc., a Division of International Paper Company, of Walpole, Mass. under the designation P-8. Examples of formed film topsheets are described in U.S. Pat. Nos. 3,929,135; 4,324,246; 4,342,314; 4,463,045; and 5,006,394. Other topsheets may be made in accordance with U.S. Pat. Nos. 4,609,518 and 4,629,643.

In some embodiments, the topsheet 188 is made of a hydrophobic material or is treated to be hydrophobic in order to isolate the wearer's skin from liquids contained in the absorbent core. If the topsheet is made of a hydrophobic material, at least the upper surface of the topsheet may be treated to be hydrophilic so that liquids will transfer through the topsheet more rapidly. The topsheet can be rendered hydrophilic by treating it with a surfactant or by incorporating a surfactant into the topsheet. A more detailed discussion of such a treatment and hydrophilicity is contained in U.S. Pat. Nos. 4,988,344 and 4,988,345. A more detailed discussion of some methods for incorporating surfactant in the topsheet can be found in U.S. Statutory Invention Registration No. H1670, which was published on Jul. 1, 1997, in the names of Aziz et al. In some embodiments, the topsheet 188 may include an apertured web or film that is hydrophobic. This may be accomplished eliminating the hydrophilizing treatment step from the production process and/or applying a hydrophobic treatment to the topsheet, such as a polytetrafluoroethylene compound like SCOTCHGUARD or a hydrophobic lotion composition, as described below. A more detailed discussion of various apertured topsheets can be found in U.S. Pat. Nos. 5,342,338; 5,941,864; 6,010,491; and 6,414,215.

The absorbent core 192 may include absorbent material that is generally compressible, conformable, non-irritating to the wearer's skin, and capable of absorbing and retaining liquids such as urine and other body exudates. The absorbent core 192 can also be manufactured in a wide variety of sizes and shapes (e.g., rectangular, hourglass, T-shaped, asymmetric, etc.). The absorbent core may also include a wide variety of liquid-absorbent materials commonly used in disposable diapers and other absorbent articles. In one example, the absorbent core includes comminuted wood pulp, which is generally referred to as airfelt. Examples of other absorbent materials include creped cellulose wadding; meltblown polymers, including coform; chemically stiffened, modified or cross-linked cellulosic fibers; tissue, including tissue wraps and tissue laminates; absorbent foams; absorbent sponges; superabsorbent polymers; absorbent gelling materials; or any other known absorbent material or combinations of materials. Exemplary absorbent structures are described in U.S. Pat. Nos. 4,610,678; 4,673,402; 4,834,735; 4,888,231; 5,137,537; 5,147,345; 5,342,338; 5,260,345; 5,387,207; and 5,650,222.

The absorbent core 192 may also have a multiple layered construction. A more detailed discussion of various types of multi-layered absorbent cores can be found in U.S. Pat. Nos. 5,669,894; 6,441,266; and 5,562,646; European Patent No. EP0565606B1; U.S. Patent Publication No. 2004/0162536A1; 2004/0167486A1; and PCT Publication No. WO 2006/015141. In some embodiments, the absorbent article includes an absorbent core that is stretchable. In such a configuration, the absorbent core may be adapted to extend along with other materials of the chassis in longitudinal and/or lateral directions. The absorbent core can also be connected with the other components of the chassis various ways. For example, the diaper may include a “floating core” configuration or a “bucket” configuration wherein the diaper includes an anchoring system that can be configured to collect forces tending to move the article on the wearer.

Although the first and second ears 156, 158 as well as the third and fourth ears 160, 162 shown in FIG. 5 are illustrated as being integrally formed with the chassis 154, it is to be appreciated that other embodiments may include ears that are discrete elements connected with the chassis. In some embodiments, the ears are configured to be stretchable. The ears may also include one or more fastener elements adapted to releasably connect with each other and/or other fastener elements on the chassis. A more detailed discussion of stretchable ears can be found in U.S. Pat. Nos. 4,857,067; 5,151,092; 5,674,216; 6,677,258; 4,381,781; 5,580,411; and 6,004,306. The ears may also include various geometries and arrangements of stretch zones or elements, such as discussed in U.S. Pat. Publication Nos. US2005/0215972A1 and US2005/0215973A1.

As shown in FIG. 5, the diaper 152 may include leg cuffs 196 that may provide improved containment of liquids and other body exudates. The leg cuffs 196 may be disposed in various ways on the diaper 152. For example, the leg cuffs 196 may be disposed on the outer, garment-facing surface 184 of the chassis 154; the inner, body-facing surface 182; or between the inner and outer facing surfaces 182 or 184. Leg cuffs 196 may also be referred to as leg bands, side flaps, barrier cuffs, or elastic cuffs. U.S. Pat. No. 3,860,003 describes a disposable diaper that provides a contractible leg opening having a side flap and one or more elastic members to provide an elasticized leg cuff (a gasketing cuff). U.S. Pat. Nos. 4,808,178 and 4,909,803 describe disposable diapers having “stand-up” elasticized flaps (barrier cuffs). U.S. Pat. Nos. 4,695,278 and 4,795,454 describe disposable diapers having dual cuffs, including gasketing cuffs and barrier cuffs. In some embodiments, it may be desirable to treat all or a portion of the leg cuffs with a lotion, as described above. In addition to leg cuffs, diaper can also include an elastic gasketing cuff with one or more elastic strands positioned outboard of the barrier cuff. The leg cuffs may be treated with a hydrophobic surface coating, such as described in U.S. Pat. Publication No. 2006/0189956A1.

The diaper may be provided in the form of a pant-type diaper or may alternatively be provided with a re-closable fastening system, which may include fastener elements in various locations to help secure the diaper in position on the wearer. For example, fastener elements may be located on the first and second ears and may be adapted to releasably connect with one or more corresponding fastening elements located in the second waist region. It is to be appreciated that various types of fastening elements may be used with the diaper. In one example, the fastening elements include hook & loop fasteners, such as those available from 3M or Velcro Industries. In other examples, the fastening elements include adhesives and/or tap tabs, while others are configured as a macrofastener or hook (e.g., a MACRO or “button-like” fastener). Some exemplary fastening elements and systems are disclosed in U.S. Pat. Nos. 3,848,594; 4,662,875; 4,846,815; 4,894,060; 4,946,527; 5,151,092; and 5,221,274. Additional examples of fasteners and/or fastening elements are discussed in U.S. Pat. Nos. 6,251,097 and 6,432,098; and U.S. Patent Publication Nos. 2007/0078427 and 2007/0093769. Other fastening systems are described in more detail in U.S. Pat. Nos. 5,595,567; 5,624,427; 5,735,840; and 5,928,212. The fastening system may also provide a means for holding the article in a disposal configuration as disclosed in U.S. Pat. No. 4,963,140.

Components of the disposable absorbent article (i.e., diaper, disposable pant, adult incontinence article, sanitary napkin, pantiliner, wipe, etc.) described in this specification can at least partially be comprised of bio-sourced content as described in US 2007/0219521A1 Hird et al. published on Sep. 20, 2007, US 2011/0139658A1 Hird, et al. published on Jun. 16, 2011, US 2011/0139657A1 Hird et al. published on Jun. 16, 2011, US 2011/0152812A1 Hird et al. published on Jun. 23, 2011, US 2011/0139662A1 Hird, et al. published on Jun. 16, 2011, and US 2011/0139659A1 Hird, et al. published on Jun. 16, 2011. These components include, but are not limited to, topsheet nonwovens, backsheet films, backsheet nonwovens, side panel nonwovens, barrier leg cuff nonwovens, super absorbent, nonwoven acquisition layers, core wrap nonwovens, adhesives, fastener hooks, and fastener landing zone nonwovens and film bases.

In at least one exemplary configuration, a disposable absorbent article component comprises a bio-based content value from about 10% to about 100% using ASTM D6866-10, method B, in another embodiment, from about 25% to about 75%, and in yet another embodiment, from about 50% to about 60% using ASTM D6866-10, method B.

In order to apply the methodology of ASTM D6866-10 to determine the bio-based content of any disposable absorbent article component, a representative sample of the disposable absorbent article component must be obtained for testing. In at least one exemplary configuration, the disposable absorbent article component can be ground into particulates less than about 20 mesh using known grinding methods (e.g., Wiley® mill), and a representative sample of suitable mass taken from the randomly mixed particles.

The absorbent article may also include discrete components 102a and 102b such as shown in FIG. 5 in the form of first and second waistbands 194a and 194b. The first and second waistbands 194a and 194b may provide improved fit and waste containment. The first and second waistbands 194a and 194b may be located in the first waist region 168 and/or the second waist region 170. The first and second waistbands 194a and 194b may be configured to elastically expand and contract to dynamically fit the wearer's waist.

The first and second waistbands 194a and 194b may extend at least longitudinally outwardly from the absorbent core 192 and generally form at least a portion of the first and/or second outer edges 178, 180 of the diaper 152. In addition, the first and second waistbands 194a and 194b may extend laterally to include the ears. While the first and second waistbands 194a and 194b or any constituent elements thereof may comprise one or more separate elements affixed to the diaper, the first and second waistbands 194a and 194b may be constructed as an extension of other elements of the diaper, such as the backsheet 190, the topsheet 188, or both the backsheet 190 and the topsheet 188. In addition, the first and second waistbands 194a and 194b may be disposed on the outer, garment-facing surface 184 of the chassis 154; the inner, body-facing surface 182; or between the inner and outer facing surfaces. It is to be appreciated that the first and second waistbands 194a and 194b shown in FIG. 5 may comprise the same materials and/or may have the same structure. While in other exemplary configurations, the first and second waistbands 194a and 194b may comprise different materials and/or may have different structures. The first and second waistbands 194a and 194b may be constructed in a number of different configurations including those described in U.S. Patent Application No. 61/499,294; and U.S. Patent Publication Nos. 2007/0142806; 2007/0142798; and 2007/0287983.

### Container

Absorbent articles of the present disclosure may be packaged in a container. A first absorbent article having a first fragrance accord may be packaged in a first container and a second absorbent article having a second fragrance accord may be packaged in a second container, wherein the first and second fragrance accords are different. The first and second containers may be placed on a store shelf for sale. The first and second absorbent articles may be offered for sale under the same brand and/or trade name.

### Examples

Example 1, shown in Table 1, is an illustrative, non-limiting formula of a fragrance accord of the present disclosure.

TABLE 1

| | | Vapor Pressure | |
|---|---|---|---|
| | | at 25° C. | Kovats |
| Perfume Raw Material Name | Weight % | (Torr) | Index |
| Prenyl Acetate | 0.20% | 4.170 | 931.3 |
| Linalool | 5.00% | 0.083 | 1159 |
| Benzyl Acetate | 4.00% | 0.187 | 1197.3 |
| Methyl Nonyl Ketone | 1.00% | 0.112 | 1297.4 |
| Iso Nonyl Acetate | 0.70% | 0.382 | 1294.4 |
| Dihydro Myrcenol | 4.00% | 0.124 | 1103.8 |
| Total Top Notes | 14.90% | | |
| Undecyl Aldehyde | 0.10% | 0.061 | 1311.6 |
| 4-Tertiary Butyl Cyclohexyl | 15.30% | 0.069 | 1351.5 |
| Acetate | | | |
| Eugenol | 20.00% | 0.009 | 1436.7 |
| Mayol | 20.00% | 0.011 | 1235.6 |
| Total Middle Notes | 55.40% | | |
| Hexyl Cinnamic Aldehyde | 5.00% | 0.001 | 1797.9 |
| Benzophenone | 2.00% | 0.001 | 1662 |
| Ethylene Brassylate | 3.00% | 0.000 | 1931 |
| Methyl Cedrylone | 2.00% | 0.001 | 1828.3 |
| Methyl Dihydro Jasmonate | 10.00% | 0.001 | 1550.9 |
| Lyral | 2.70% | 8.57%-05 | 1587 |
| Benzyl Salicylate | 5.00% | 0.000 | 1920.3 |
| Total Base Notes | 29.70% | | |
| TOTAL | 100.00% | | |

Example 2, shown in Table 2, is an illustrative, non-limiting formula of a fragrance accord of the present disclosure.

TABLE 2

| | | Vapor Pressure at | |
|---|---|---|---|
| | | 25° C. | Kovats |
| Perfume Raw Material Name | Weight % | (Torr) | Index |
| Prenyl Acetate | 1.00% | 4.170 | 931.3 |
| Linalool | 5.00% | 0.083 | 1159 |
| Benzyl Acetate | 4.00% | 0.187 | 1197.3 |
| Methyl Nonyl Ketone | 1.00% | 0.112 | 1297.4 |
| Iso Nonyl Acetate | 0.70% | 0.382 | 1294.4 |
| Dihydro Myrcenol | 8.00% | 0.124 | 1103.8 |
| Total Top Notes | 19.70% | | |
| Undecyl Aldehyde | 0.20% | 0.061 | 1311.6 |
| 4-Tertiary Butyl Cyclohexyl | 5.00% | 0.069 | 1351.5 |
| Acetate | | | |
| Eugenol | 10.00% | 0.009 | 1436.7 |
| Mayol | 5.00% | 0.011 | 1235.6 |
| Total Middle Notes | 20.20% | | |
| Hexyl Cinnamic Aldehyde | 15.00% | 0.001 | 1797.9 |
| Benzophenone | 2.00% | 0.001 | 1662 |
| Ethylene Brassylate | 10.10% | 0.000 | 1931 |
| Methyl Cedrylone | 5.00% | 0.001 | 1828.3 |
| Methyl Dihydro Jasmonate | 20.00% | 0.001 | 1550.9 |
| Lyral | 3.00% | 8.57%-05 | 1587 |
| Benzyl Salicylate | 5.00% | 0.000 | 1920.3 |
| Total Base Notes | 60.10% | | |
| TOTAL | 100.00% | | |

Example 3, shown in Table 3, is an illustrative, non-limiting formula of a fragrance accord of the present disclosure.

TABLE 3

| | | Vapor Pressure at | |
|---|---|---|---|
| | | 25° C. | Kovats |
| Perfume Raw Material Name | Weight % | (Torr) | Index |
| Prenyl Acetate | 3.00% | 4.170 | 931.3 |
| Linalool | 15.90% | 0.083 | 1159 |
| Benzyl Acetate | 15.00% | 0.187 | 1197.3 |
| Methyl Nonyl Ketone | 1.00% | 0.112 | 1297.4 |
| Iso Nonyl Acetate | 5.00% | 0.382 | 1294.4 |
| Dihydro Myrcenol | 20.00% | 0.124 | 1103.8 |
| Total Top Notes | 59.90% | | |
| Undecyl Aldehyde | 0.10% | 0.061 | 1311.6 |
| 4-Tertiary Butyl Cyclohexyl | 3.00% | 0.069 | 1351.5 |
| Acetate | | | |
| Eugenol | 3.00% | 0.009 | 1436.7 |
| Mayol | 5.00% | 0.011 | 1235.6 |
| Total Middle Notes | 11.10% | | |
| Hexyl Cinnamic Aldehyde | 5.00% | 0.001 | 1797.9 |
| Benzophenone | 1.00% | 0.001 | 1662 |
| Ethylene Brassylate | 3.00% | 0.000 | 1931 |
| Methyl Cedrylone | 2.00% | 0.001 | 1828.3 |
| Methyl Dihydro Jasmonate | 10.00% | 0.001 | 1550.9 |
| Lyral | 3.00% | 8.57%-05 | 1587 |
| Benzyl Salicylate | 5.00% | 0.000 | 1920.3 |
| Total Base Notes | 29.00% | | |
| TOTAL | 100.00% | | |

Kovats Index Method

Kovats Index is determined by gas chromatography. An Agilent Technologies 6890 gas chromatogram equipped with a DB-5 column and a Flame Ionization Detector (FID), or equivalent instrument with FID, is used. A suitable column is 30 m×0.25 mm i.d., with a 0.25 um film. The carrier gas is helium. Gas flow rate, injection temperature and column temperature are adjusted to achieve good separation of peaks. For example; helium flow rate from 1 to 3 mL/min; inlet temperature 280° C.; column temperature start at 40° C., ramp to 90° C. at 25° C./min, then ramp to 170° C. at 15° C./min, then hold at 170° C. for 2 min. Different conditions may be required depending on the nature of the column and analytes.

A linear alkane hydrocarbon standard mix (C8-C22) available from Aldrich Co., AccuStandard Co., or equivalent is injected and the retention time for each component is noted. The test sample (appropriately diluted in a suitable solvent) is injected under the same conditions, and the retention time for each component is noted as well as the area under each peak. The retention time for each peak in the test sample will lie between the retention times of two alkanes in the standard mix.

Kovats Index I is calculated for each component in the test sample using the equation:

$$I = 100 \times [ n + ( N - n ) \frac{log ( {t}_{r ( unknown )}^{′} ) - log ( {t}_{r ( n )}^{′} )}{log ( {t}_{r ( N )}^{′} ) - log ( {t}_{r ( n )}^{′} )} ]$$

Where I is the Kovats Index, n is the number of carbon atoms in the smaller alkane whose peak is adjacent to that of the test component, N is the number of carbon atoms in the larger alkane whose peak is adjacent to that of the test component, t′ᵣ is the retention time.

The FID Area Fraction (AFᵢ) is determined for each component in the test sample as a fraction of the total integrated peak area for all components in the test sample. The FID Area Fraction of each component is taken as the mass fraction of that component in the test sample.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. An array of wet wipes products comprising:
(a) a first wet wipes product having a first fragrance accord, the first fragrance accord comprising:
from about 10% to about 15%, by total weight, of a perfume raw material having a vapor pressure of greater than 0.08 Torr at 25° C.;
from about 35% to about 65%, by total weight, of a perfume raw material having a vapor pressure from 0.006 Torr at 25° C. to 0.08 Torr at 25° C.; and
from about 20% to about 30%, by total weight, of a perfume raw material having a vapor pressure of less than 0.006 Torr at 25° C.; and
(b) a second wet wipes product having a second fragrance accord, the second fragrance accord comprising:
from about 10% to about 20%, by total weight, of a perfume raw material having a vapor pressure of greater than 0.08 Torr at 25° C.;
from about 20% to about 30%, by total weight, of a perfume raw material having a vapor pressure from 0.006 Torr at 25° C. to 0.08 Torr at 25° C.; and
from about 45% to about 70%, by total weight, of a perfume raw material having a vapor pressure of less than 0.006 Torr at 25° C.

2. The array of claim 1, wherein the first wet wipes product comprises a first lotion composition and the second wet wipes product comprises a second lotion composition, wherein the first lotion composition comprises the first fragrance accord, wherein the second lotion composition comprises the second fragrance accord.

3. The array of claim 1, wherein each of the first and second wet wipes products comprises a non-woven substrate.

4. The array of claim 1, wherein at least one of the first and second fragrance accords is neat.

5. The wet wipe of claim 2, wherein at least one of the first and second lotion compositions is aqueous.

6. The wet wipe of claim 2, wherein at least one of the first and second lotion compositions comprises an emollient.

7. The wet wipe of claim 6, wherein the emollient comprises an oil material.

8. The wet wipe of claim 3, wherein the substrate comprises a co-form structure.

9. The wet wipe of claim 3, wherein the substrate comprises a layered structure.

10. An array of wet wipes products comprising:
(a) a first wet wipes product having a first fragrance accord, the first fragrance accord comprising:
from about 10% to about 15%, by total weight, of a perfume raw material having a Kovats Index of less than 1300;
from about 35% to about 65%, by total weight, of a perfume raw material having a Kovats Index from 1300 to 1450; and
from about 20% to about 30%, by total weight, of a perfume raw material having a Kovats Index of greater than 1450; and
(b) a second wet wipes product having a second fragrance accord, the second fragrance accord comprising:
from about 10% to about 20%, by total weight, of a perfume raw material having a Kovats Index of less than 1300;
from about 20% to about 30%, by total weight, of a perfume raw material having a Kovats Index from 1300 to 1450; and
from about 45% to about 70%, by total weight, of a perfume raw material having a Kovats Index of greater than 1450.

11. The array of claim 10, wherein the first wet wipes product comprises a first lotion composition and the second wipes product comprises a second lotion composition, wherein the first lotion composition comprises the first fragrance accord, wherein the second lotion composition comprises the second fragrance accord.

12. The array of claim 10, wherein each of the first and second wet wipes products comprises a non-woven substrate.

13. The wet wipe of claim 10, wherein at least one of the first and second fragrance accords is neat.

14. The wet wipe of claim 11, wherein at least one of the first and second lotion compositions is aqueous.

15. The wet wipe of claim 11, wherein at least one of the first and second lotion compositions comprises an emollient.

16. The wet wipe of claim 15, wherein the emollient comprises an oil material.

17. The wet wipe of claim 12, wherein the substrate comprises a co-form structure.

18. The wet wipe of claim 12, wherein the substrate comprises a layered structure.
